(19) United States
(12) Patent Application Publication
(10) Pub. No.: US 2009/0298803 A1
(43) Pub. Date: Dec. 3, 2009

(54) PHARMACEUTICAL COMPOSITION COMPRISING FLUOCINONIDE

(75) Inventors: Nilendu Sen, Mumbai (IN); Amol Mandhare, Maharashtra (IN); Shantesh Patil, Maharashtra (IN)

Correspondence Address: GLENMARK PHARMACEUTICALS INC USA, 750 CORPORATE DRIVE, MAHWAH, NJ, 07430, US

(73) Assignee: GLENMARK GENERICS LTD., Mumbai (IN)

(21) Appl. No.: 12/494,356

(22) Filed: Jun. 30, 2009

Related U.S. Application Data
(60) Provisional application No. 61/163,159, filed on Mar. 25, 2009.

(30) Foreign Application Priority Data
May 28, 2008 (IN) 1141/MUM/2008

(51) Int. Cl. A61K 31/58; A61P 29/00

(52) U.S. Cl. 514/174

(57) ABSTRACT

The present invention provides a composition, comprising fluocinonide in a vehicle comprising one or two penetration enhancers, solvents and emulsifiers wherein penetration enhancers are present in ratio of less than 0.90 to the total of the penetration enhancers, solvents and emulsifiers.

### PRIORITY

This application claims the benefit under 35 U.S.C. §119 to Indian Provisional Application 1141/MUM/2008 filed on May 28, 2008 and to U.S. Provisional Application 61/163159 filed on Mar. 25, 2009, the contents of each of which, are incorporated by reference herein.

### BACKGROUND OF THE INVENTION

1. Technical Field

The present invention comprises a composition, comprising fluocinonide in a vehicle comprising one or two penetration enhancers, solvents and emulsifiers, wherein the penetration enhancers are present in a ratio of less than 0.90 relative to the total of the penetration enhancers, solvents and emulsifiers.

2. Description of Related Art

Fluocinonide, with the chemical structure as shown below, is a synthetic corticosteroid for topical dermatologic use. The corticosteroids constitute a class of primarily synthetic steroids, which are used topically as anti-inflammatory and antipruritic agents. Fluocinonide has the chemical name 6-α, 9-α-difluoro-11-β, 21-dihydroxy-16-α, 17-α-isopropylidenedioxypregna-1,4-diene-3,20-dione-21-acetate. Its chemical formula is C₂₆H₃₂F₂O₇ with a molecular weight of 494.58.

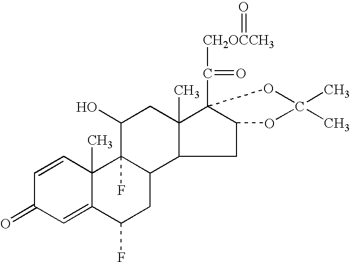

The extent of percutaneous absorption of topical corticosteroids is determined by many factors including the vehicle and the integrity of the epidermal barrier. Topical corticosteroids can be absorbed from normal intact skin. Inflammation and/or other disease processes in the skin may increase percutaneous absorption.

U.S. Pat. No. 6,765,001 (the '001 patent) discloses a composition of fluocinonide with increased vasoconstrictor potency. The '001 patent discloses a composition, which comprises two or more penetration enhancers selected from the group consisting of diisopropyl adipate, dimethyl isosorbide, propylene glycol, 1,2,6-hexapetriol, and benzyl alcohol; and one or more of the group consisting of solvents and emulsifiers, wherein the penetration enhancers are present in a ratio to a total of the penetration enhancers, and solvents and emulsifiers of at least about 0.90.

### SUMMARY OF THE INVENTION

One aspect of the invention is a topical cream comprising about 0.005 wt % to about 1.0 wt % fluocinonide, or a pharmaceutically acceptable salt or ester thereof and a penetration enhancer.

Another aspect of the present invention is a topical fluocinonide cream for the treatment of skin conditions (i.e., deriatological disorders). The cream comprises about 0.005 wt % to 1.0 wt % fluocinonide, or a pharmaceutically acceptable salt or ester thereof; most preferably 0.1%; a penetration enhancer, solvent and emulsifier with other auxiliary substances. The cream optionally contains additives such as preservatives and buffers.

Another aspect of the invention is a topical cream comprising fluocinonide in an amount of from about 0.1 wt. %; a penetration enhancer, in an amount of from about 60.0 wt % to about 80.0 wt %; solvent emulsifier in an amount of from about 5.0 wt % to about 20.0 wt %; and other auxiliary substances.

Further, another aspect of the present invention provides topical cream composition comprising fluocinonide with one or more penetration enhancers and one or more solvent emulsifiers, wherein the ratio to the total of the penetration enhancers, solvents emulsifiers is less than about 0.90.

### DETAILED DESCRIPTION OF INVENTION

Fluocinonide, or a pharmaceutically acceptable salt or ester thereof, is present in the formulation in a concentration of from about 0.005 wt % to about 1.0 wt. %, preferably from about 0.01 wt % to about 0.1 wt %, and one or more penetration enhancers and one or more solvent emulsifiers wherein ratio is less than about 0.90 to the total of the, penetration enhancers, and solvents emulsifiers.

Prior art compositions deal with compositions, which require two or more penetration enhancers and the ratio limited to at least 0.90 to the total of the penetration enhancers, solvents and emulsifiers in the composition.

The present invention provides that at least one conventional penetration enhancer may be used in the cream composition. For example, the penetration enhancer may include, but are not limited to, propylene glycol, diisopropyl adipate, dimethyl isosorbide, 1,2,6 hexanetriol, and benzyl alcohol. The penetration enhancer may be present in a concentration in the range of about 60.0 wt % to about 80.0 wt %, preferably about 70.0 wt % to about 75.0 wt %.

The preferred penetration enhancer used in the cream composition of the present invention is propylene glycol, at a concentration of from about 60.0 wt % to about 80.0 wt %,.

The present invention provides the use of solvent emulsifiers in the cream composition, which may include one or more of dehydrated alcohol, alcohol (95% v/v) USP, 3-cyclohexene-1-cethanol, 4-dimethyl-a-(4-methyl-3-pentenyl)-, steareth-2, steareth-21, citric acid, CPE®-215, diisopropanolamine (1:9), DIPA/PG (1:9), diethylene glycol monoethyl ether (Transcutol® P), potassium hydroxide (10%), polyethylene glycol (PEG)-40 Stearate, PEG-7000, polysorbate 60, potassium hydroxide (1%), propylene carbonate USP, propylethylene glycol 4, oleyl alcohol, sodium lauryl sulfate, sorbitan monostearate, sorbitan stearate, and 1,2,3-propanetriol ester.

The composition, described herein above, optionally comprises other auxiliary substances such as glyceryl stearate (and) PEG-100 stearate; Carbopol® 980, cyclomethicone NF, glyceryl monostearate, hydroxyethyl cellulose, hydroxypropyl cellulose, isopropyl myristate, methyl paraben NF, mineral oil, oleic acid NF, PEG-100 stearate, petrolatum, purified water, stearyl alcohol, white petrolatum, and white wax.

Optionally, conventional preservatives may be used in the composition, herein described. Preferably, preservatives employed in the formulation should pass US Pharmacopoeia, British Pharmacopoeia and European Pharmacopoeia standards. Preferred preservatives include, but are not limited to, imidurea, methylparaben, propylparaben and the like, and combinations thereof.

As used herein, the term “stable” or “substantially stable” refers to an active compound which remains within about ±10%, preferably about 6%, by weight, of the original amount, when incubated at the recited temperature for the recited amount of time in a closed container.

Treatment of skin conditions with the cream described herein is accomplished by applying the cream composition to the affected areas to be treated. The treatment regimen is varied from patient to patient and condition to condition. Preferably, the cream of the present invention is used to treat atopic dermatitis, inflammatory and pruritic manifestations and corticosteroid-responsive dermatoses.

The cream of the present invention is manufactured in a conventional manner by mixing the ingredients at elevated temperatures (such as from about 45° C. to about 80° C.) and then cooling the mixture to achieve a smooth, homogeneous cream composition.

The following examples merely illustrate the compositions of the invention and are not to be construed as limiting the scope of the invention. Unless indicated otherwise, all weight percentages are based on the total weight of the composition

### EXAMPLES

A topical 0.1 wt. % fluocinonide cream in accordance with the present invention was prepared having the following composition.

### Example 1

| Sr. | | concentration |
|---|---|---|
| No. | Ingredients | % w/w |
| 1. | Fluocinonide | 00.1 |
| 2. | Dimethyl isosorbide | 5.0 |
| 3. | Diethylene Glycol Monoethyl Ether | 10.0 |
| | (Transcutol P) | |
| 4. | Propylene Glycol | 70.0 |
| 5. | Carbomer 940 (Carbopol 980 NF) | 1.2 |
| 6. | Glyceryl Stearate/PEG 100 Stearate | 7.5 |
| | (Arlacel 165) | |
| 7. | Glyceryl Monostearate | 2.5 |
| 8. | Diisopropanolamine 85% | 1.2 |
| | (1:9 with Propylene Glycol) | |
| 9. | Purified Water | 2.49 |
| 10. | Citric Acid | 0.01 |

Procedure:
1. Mixed well Dimethyl isosorbide and diethyleneglycol monoethylether (Transcutol P). Then added fluocinonide into it and dissolved completely.
2. Carbomer® 940 (Carbopol® 980 NF) was added to propylene glycol and stirred until homogeneous Carbomer® 940 (Carbopol®980 NF) dispersion is formed.
3. Citric acid was added to purified water and stirred to dissolve completely.
4. Added 3 to 2 and mixed well.
5. Glyceryl stearate/PEG 100 Stearate (Arlacel® 165) and glyceryl monostearate were taken and heated to 65° C. to 70° C. and melted completely.
6. Heated the mixture prepared in 4, to 65° C. to 70° C.
7. Transferred mixture prepared in 5 to 6 under stirring and mixed for 15 minutes. After cooling to temperature 50° C. to 55° C. added solution prepared in 1 and mixed for 15 minutes.
8. Then added diisopropanolamine 85% ((1:9 with propylene glycol) and mixed well.
9. Then cooled to room temperature.

### Example 2

| Sr. | | concentration |
|---|---|---|
| No. | Ingredients | % w/w |
| 1. | Fluocinonide | 00.1 |
| 2. | Dimethyl isosorbide | 5.0 |
| 3. | Diethylene Glycol Monoethyl ether | 5.0 |
| | (Transcutol ® P) | |
| 4. | Propylene Carbonate | 5.0 |
| 5. | Propylene Glycol | 70.0 |
| 6. | Carbomer ® 940 | 1.2 |
| | (Carbopol ® 980 NF) | |
| 7. | Glyceryl Stearate/PEG 100 Stearate | 7.5 |
| | (Arlacel ® 165) | |
| 8. | Glyceryl Monostearate | 2.5 |
| 9. | Diisopropanolamine 85% | 1.2 |
| | (1:9 with Propylene Glycol) | |
| 10. | Purified Water | 2.49 |
| 11. | Citric Acid | 0.01 |

Procedure:
1. Mix well dimethyl isosorbide, diethylene glycol monoethyl ether (Transcutol® P) and propylene carbonate. Then added fluocinonide into it and dissolved completely.
2. Carbomer®940 (Carbopol®980 NF) was added to propylene glycol and stirred until homogeneous Carbomer® 940 (Carbopol® 980 NF) dispersion is formed.
3. Citric acid was added to purified water and stirred to dissolve completely.
4. Added 3 to 2 and mixed well.
5. Glyceryl stearate/PEGI00 stearate (Arlacel® 165) and glyceryl monostearate were heated to 65° C. to 70° C. and melted completely.
6. Heated the mixture prepared in 4, to 65° C. to 70° C.
7. Transferred mixture prepared in 5 to 6 under stirring and mixed for 15 minutes. After cooling to temperature 50° C. to 55° C. added solution prepared in 1 and mixed for 15 minutes.
8. Then added diisopropanolamine 85% ((1:9 with propylene glycol) and mixed well.
9. Then cooled to room temperature.

### Example 3

| Sr. | | concentration |
|---|---|---|
| No. | Ingredients | % w/w |
| 1. | Fluocinonide | 00.1 |
| 2. | Diethylene Glycol Monoethyl ether | 15.0 |
| | (Transcutol ® P) | |
| 3. | Propylene Glycol | 70.0 |
| 4. | Carbomer ® 940 (Carbopol ® 980 NF) | 1.2 |
| 5. | Glyceryl stearate/PEG 100 Stearate | 7.5 |
| | (Arlacel ® 165) | |
| 6. | Glyceryl monostearate | 2.5 |
| 7. | Diisopropanolamine 85% | 1.2 |
| | (1:9 with Propylene Glycol) | |
| 8. | Purified Water | 2.49 |
| 9. | Citric Acid | 0.01 |

Procedure:
1. Added fluocinonide into diethylene glycol monoethyl ether (Transcutol®P) and dissolved completely.
2. Carbomer®940 (Carbopol®980 NF) was added to propylene glycol and stirred until homogeneous Carbomer®940 (Carbopol®980 NF) dispersion is formed.
3. Citric acid was added to purified water and stirred to dissolve completely.
4. Transferred 3 to 2 and mixed well.
5. Glyceryl stearate/PEG 100 stearate (Arlacel®165) and glyceryl monostearate were heated to 65° C. to 70° C. and melted completely.
6. Heated mixture prepared in 4 to 65° C. to 70° C.
7. Transferred mixture prepared in 5 to 7 under stirring and mixed for 15 minutes. After cooling to temperature 50° C. to 55° C. added solution prepared in I and mixed for 15 minutes.
8. Then added diisopropanolamine 85% ((1:9 with propylene glycol) and mixed well.
9. Then cooled to room temperature.

### Example 4

| Sr. | | Concentration |
|---|---|---|
| No. | Ingredients | % w/w |
| 1. | Fluocinonide | 00.1 |
| 2. | Diethylene Glycol Monoethyl ether | 10.0 |
| | (Transcutol ® P) | |
| 3. | Propylene Carbonate | 5.0 |
| 4. | Propylene Glycol | 70.0 |
| 5. | Carbomer ® 940 | 1.2 |
| | (Carbopol ® 980 NF) | |
| 6. | Glyceryl Stearate/PEG 100 Stearate | 7.5 |
| | (Arlacel ® 165) | |
| 7. | Glyceryl Monostearate | 2.5 |
| 8. | Diisopropanolamine 85% | 1.2 |
| | (1:9 with Propylene Glycol) | |
| 9. | Purified Water | 2.49 |
| 10. | Citric Acid | 0.01 |

Procedure:
1. Mixed well diethyleneglycolmonoethyl ether (Transcutol®P) and propylene carbonate. Then added fluocinonide into it and dissolved completely.
2. Carbomer®940 (Carbopol®980 NF) was added to propylene glycol and stirred until homogeneous Carbomer®940 (Carbopol®980 NF) dispersion is formed.
3. Citric acid was added to purified water and stirred to dissolve completely.
4. Added 3 to 2 and mixed well.
5. Glyceryl stearate/PEG100 stearate (Arlacel®165) and glyceryl monostearate were heated to 65° C. to 70° C. and melted completely.
6. Heated mixture prepared in 4, to 65° C. to 70° C.
7. Transferred mixture prepared in 5 to 7 under stirring and mixed for 15 minutes. After cooling to temperature 50° C. to 55° C. added solution prepared in 1 and mixed for 15 minutes.
8. Then added diisopropanolamine 85% ((1:9 with propylene glycol) and mixed well.
9. Then cooled to room temperature.

### Vasoconstriction Study

A vasoconstriction study was performed with VANOS® Cream, 0.1% and test sample from Example 3, in 36 qualified healthy subjects, the results of said vasoconstriction studies are provided in Table 5.

TABLE 5

| | Summed | |
|---|---|---|
| Sample | Vasoconstriction Scores | AUEC |
| VANOS ® Cream (R) | 575.03 | 15.9731 |
| EXAMPLE 3 (Test) | 583.32 | 16.2033 |

The ratio of area under effective curve (AUEC) for Test (composition of Example 3) and reference composition (VANOS® Cream 0.1%) was found to be 101.44, which suggests that composition described herein above, is comparable to the marketed product with respect to the vasoconstriction properties.

We claim:

1. A topical cream composition comprising fluocinonide, or a pharmaceutically acceptable salt or ester thereof from about 0.005 wt % to about 1.0 wt %, and a single penetration enhancer.

2. The composition of claim 1, where the penetration enhancer is from about 60.0 wt % to about 80.0 wt %.

3. The composition of claim 1, wherein the penetration enhancer is selected from propylene glycol, diisopropyl adipate, dimethyl isosorbide, 1,2,6-hexanetriol, and benzyl alcohol.

4. The composition of claim 3, wherein the penetration enhancer is propylene glycol.

5. A topical cream composition comprising of fluocinonide, or a pharmaceutically acceptable salt or ester thereof from about 0.005 wt % to about 1.0 wt %, a single penetration enhancer, solvent-emulsifiers and other auxiliary substances.

6. The composition of claim 5, wherein the solvent-emulsifiers are selected from the group comprising of dehydrated alcohol, alcohol (95% v/v) USP, 3-cyclohexene-1-cethanol, 4-dimethyl-a-(4-methyl-3-pentenyl)-, steareth-2, steareth-21, citric acid, CPE®-215, diisopropanolamine (1:9), DIPA/PG (1:9), diethyleneglycol monoethylether (TRANSCUTOL® P), potassium hydroxide (10%), polyethylene glycol (PEG)-40 Stearate, PEG-7000, polysorbate 60, potassium hydroxide (1%), propylene carbonate USP, propylethylene glycol 4, oleyl alcohol, sodium lauryl sulfate, sorbitan monostearate, sorbitan stearate, and 1,2,3-propanetriol ester.

7. The composition of claim 6, wherein the solvent emulsifier is diethyleneglycol monoethylether.

8. The composition of claim 5, wherein the other auxiliary substances are selected from the group of glyceryl stearate (and) PEG-100 stearate, CARBOPOL® 980, cyclomethicone NF, glyceryl monostearate, hydroxyethyl cellulose, hydroxypropyl cellulose, isopropyl myristate, methyl paraben NF, mineral oil, oleic acid NF, PEG-100 stearate, petrolatum, purified water, stearyl alcohol, white petrolatum, and white wax.

9. The composition of claim 5 comprising of 0.1 wt % fluocinonide; 70.0 wt % to about 75.0 wt % propylene glycol; 0-20.0 wt % diethyleneglycol monoethylether; 0.5-2.0 wt % CARBOMER®940.

10. The composition of claim 5, wherein ratio of the penetration enhancers and solvent-emulsifiers is less than 0.90.

11. A composition comprising about 0.1 wt % fluocinonide; about 71.08 wt % propylene glycol; about 15.0 wt % diethyleneglycol monoethylether; about 1.2wt % CARBOMER ®940; about 7.5 wt % glyceryl stearate/PEG100 stearate; about 2.5 wt % glyceryl monostearate; about 0.12wt % diisopropanolamine and about 0.01 wt % citric acid, which is a topical cream.
